(19) United States
(12) Patent Application Publication
(10) Pub. No.: US 2017/0082594 A1
(43) Pub. Date: Mar. 23, 2017

(54) METHODS FOR REDUCING THE PRECIPITATION PROPENSITY OF ASPHALTENES IN BLENDED CRUDE OILS

(71) Applicant: FLINT HILLS RESOURCES, LP, Wichita, KS (US)

(72) Inventors: Scott Blumenshine, Wichita, KS (US); Ryan Jilka, Wichita, KS (US)

(73) Assignee: FLINT HILLS RESOURCES, LP, Wichita, KS (US)

(21) Appl. No.: 15/310,880

(22) PCT Filed: May 15, 2015
(86) PCT No.: PCT/US15/31197

§ 371 Date: Nov. 14, 2016

Related U.S. Application Data
(60) Provisional application No. 61/993,308, filed on May 15, 2014.

(51) Int. Cl. G01N 33/28; G01N 21/359; G01N 24/08; G01N 21/3577

(52) U.S. Cl. CPC G01N 33/2823; G01N 21/3577; G01N 21/359; G01N 24/08

(57) ABSTRACT

Methods for selecting crude oils to prepare a crude oil blend. A precipitation propensity of a first crude oil and a precipitation propensity of at least one second crude oil can be measured. At least one of the first crude oil and the second crude oil can include asphaltenes. The precipitation propensity of the first crude oil can be multiplied by a proposed volume percent of the first crude oil in a proposed crude oil blend to provide a first value. The precipitation propensity of the second crude oil can be multiplied by a proposed volume percent of the second crude oil in the proposed crude oil blend to provide a second value. A composition of the proposed crude oil blend can be modified if a sum of the first value and the second value is less than a predetermined cutoff.

### CROSS-REFERENCE TO RELATED APPLICATION

This application claims priority to U.S. Provisional Patent Application No. 61/993,308, filed on May 15, 2014, which is incorporated by reference herein.

### BACKGROUND

Field

Embodiments described generally relate to methods for reducing the precipitation propensity of asphaltenes in blended crude oils.

Description of Related Art

When certain crude oils are blended, when crude oils are blended in certain proportions, and/or when crude oils are blended in a certain order, asphaltenes can aggregate, precipitate, and/or flocculate from the crude oil blends, which is generally referred to as incompatibility, with potential technical and environmental consequences. Asphaltene aggregation problems can be encountered during storage, transport, and processing of the crude oil blend and it can be difficult to predict when, where, and to what extent such problems will arise. For example, precipitated asphaltenes are difficult to re-suspend quickly and can lead to rapid fouling of preheat exchangers and/or rapid coking of crude unit heaters, for example. The precipitation of asphaltenes can also cause reservoir plugging, fouling of product facilities, alterations in wettability, an increase in waste water treatment demands, and poisoning of refinery catalysts.

Asphaltenes are defined as the fraction of crude oil that is insoluble in light n-alkanes, e.g., n-heptane, but soluble in aromatic solvents, e.g., toluene. See Mitchell, D. L. et al., Fuel, 52(4):149-152 (1973) and Carnahan, N. and J. L. Salager Raquel Anton, Effect of resins on stability of asphaltenes, offshore Technology Conference, Houston, Tex., U.S.A, 30 Apr.-3 May 2007. Changes in pressure, temperature, and/or composition during the production, transport, and/or refining stages can cause the formation of asphaltenes. See, e.g., Hirschberg, A., et al., SPE J., June: 283-292 (1984); Andersen, et al., Petrol. Sci. Technol., 16:323-334; (1998); Fenistein, D., et al., Langmuir, 145:1013-1020 (1998); and Vafaie-Sefti, M. et al., Fluid Phase Equilibria, 247:182-189 (2006). Measuring the flocculation onset of crude oil blends plays an important role in avoiding asphaltene deposition. See, e.g., Wang et al., A Practical Method for Anticipating Asphaltene Problems, SPE Production & Facilities (2004). Although most crude oils are compatible to blend, the number of incompatible crude pairs exceeds 500 and is climbing because of an increase in the purchase of “opportunity crude oils” for improving refinery economics. As the number of incompatible crude pairs increase, the frequency of incompatible crude oil blends and the potential technical and environmental consequences escalate.

There is a need, therefore, for improved methods for reducing the precipitation propensity of asphaltenes in blended crude oils.

### SUMMARY

Methods for selecting crude oils to prepare a crude oil blend are provided. In some examples, the method can include measuring a precipitation propensity of a first crude oil and measuring a precipitation propensity of at least one second crude oil. At least one of the first crude oil and the second crude oil can include asphaltenes. The precipitation propensity of the first crude oil can be multiplied by a proposed volume percent of the first crude oil in a proposed crude oil blend to provide a first value. The precipitation propensity of the second crude oil can be multiplied by a proposed volume percent of the second crude oil in the proposed crude oil blend to provide a second value. A composition of the proposed crude oil blend can be modified if a sum of the first value and the second value is less than a predetermined cutoff.

In other examples, the method for selecting crude oils to prepare a crude oil blend can include proposing an amount of a first crude oil and an amount of at least one second crude oil for combining with one another to produce a proposed crude oil blend. At least one of the first crude oil and the second crude oil can include asphaltenes. A precipitation propensity of the first crude oil and the second crude oil can be measured. The precipitation propensity of the first crude oil can be multiplied by a volume percent of the first crude oil in the proposed crude oil blend to provide a first value. The precipitation propensity of the second crude oil can be multiplied by a volume percent of the second crude oil in the proposed crude oil blend to provide a second value. The first value and the second value can be added to provide a sum for the proposed crude oil blend. The method can also include combining the first crude oil and the second crude oil with one another to produce the proposed crude oil blend or modifying a composition of the proposed crude oil blend to produce a modified proposed crude oil blend. For example, the first crude oil and the second crude oil can be combined with one another to produce the proposed crude oil blend if the sum for the proposed crude oil blend is equal to or greater than a predetermined cutoff. In another example, a composition of the proposed crude oil blend can be modified if the sum for the proposed crude oil blend is less than the predetermined cutoff to produce a modified proposed crude oil blend. A sum for the modified proposed crude oil blend can be equal to or greater than the predetermined cutoff.

In other examples, the method for selecting crude oils to prepare a crude oil blend can include proposing an amount of a first crude oil and an amount of at least one second crude oil for combining with one another to produce a proposed crude oil blend. At least one of the first crude oil and the second crude oil can include asphaltenes. A solubility ratio of the first crude oil and a solubility ratio of the second crude oil can be measured. Measuring the solubility ratio for each of the first crude oil and the second crude oil can include adding to a volume of each of the first crude oil and the second crude oil a normal paraffin. A volume of the normal paraffin that causes a minimum optical density by near infrared spectroscopy for each of the first crude oil and the second crude oil can be measured. The volume of the asphaltene non-solvent that causes the minimum optical density in the first crude oil can be divided by the volume of the first crude oil to provide the solubility ratio of the first crude oil. The volume of the asphaltene non-solvent that causes the minimum optical density in the second crude oil can be divided by the volume of the second crude oil to provide the solubility ratio of the at least one second crude oil. The solubility ratio of the first crude oil can be multiplied by a volume percent of the first crude oil in the proposed crude oil blend to provide a first value. The solubility ratio of the second crude oil can be multiplied by a volume percent of the second crude oil in the proposed crude oil blend to provide a second value. The first value and the second value can be added to provide a sum for the proposed crude oil blend. The method can also include combining the first crude oil and the second crude oil with one another to produce the proposed crude oil blend or modifying a composition of the proposed crude oil blend to produce a modified proposed crude oil blend. For example, the first crude oil and the second crude oil can be combined with one another to produce the proposed crude oil blend if the sum for the proposed crude oil blend is equal to or greater than a predetermined cutoff. In another example, a composition of the proposed crude oil blend can be modified if the sum for the proposed crude oil blend is less than the predetermined cutoff to produce a modified proposed crude oil blend. A sum for the modified proposed crude oil blend can be equal to or greater than the predetermined cutoff.

### BRIEF DESCRIPTION OF THE DRAWINGS

FIG. 1 depicts a schematic of an illustrative instrument set-up, including a near infrared spectrometer, according to one or more embodiments described.

FIG. 2 is a graphical depiction of an experimental plot of optical density as a function of the volume of n-heptane during titration of a crude oil, as measured using near infrared spectrometry.

### DETAILED DESCRIPTION

As employed above and throughout the disclosure, the following terms, unless otherwise indicated, shall be understood to have the following meanings.

As used herein, the singular forms “a,” “an,” and “the” include the plural reference unless the context clearly indicates otherwise.

Crude oil components can be classified according to their polarizability and polarity into saturates, aromatics, resins, and asphaltenes. Saturates contain nonpolar material including linear, branched, and cyclic saturated hydrocarbons, e.g., paraffins. Aromatics, which contain one or more aromatic rings, are slightly polarizable. The remaining two fractions, resins and asphaltenes, have polar substituents. The distinction between the two is that asphaltenes are insoluble in an excess of heptane (or pentane), whereas resins are miscible with heptane (or pentane).

As used herein, the term “asphaltene” refers to a class of hydrocarbons in carbonaceous material, such as crude oil, bitumen, or coal that is soluble in toluene, xylene, and benzene, yet insoluble in n-alkanes, e.g., n-heptane and n-pentane. Asphaltenes are generally characterized by fused ring aromaticity with some small aliphatic side chains, and typically some polar heteroatom-containing functional groups, e.g., carboxylic acids, carbonyl, phenol, pyrroles, and pyridines, capable of donating or accepting protons intermolecularly and/or intramolecularly, having a molar H/C ratio of about 1 to 1.2, and a N, S, and O content of a low weight percent.

As used herein, the term “precipitation propensity” refers to the tendency of a composition that includes a first crude oil or a composition that includes a first crude oil and at least one second crude oil to precipitate asphaltenes, where at least one of the first crude oil and the second crude oil includes asphaltenes. The precipitation propensity can be measured by any conventional technique for measuring asphaltene precipitation or aggregation, including, but not limited to, volumetric solvent titrimetry with optical measurement, e.g., infrared spectroscopy and/or near infrared spectroscopy, including oil compatibility models; Asphaltene Stability Index (ASI) test using solvent-titration, as described in Gawrey, et al., Instrumentation Science & Technology, 2004, 32(3), 247-253; solvent titrimetry with electrical measurement, e.g., conductivity and/or capacitance, as described in U.S. patent No.: U.S. Pat. No. 5,420,040; solvent titrimetry with surface tension measurement, as described in U.S. Pat. No. 5,420,040; spot testing, as described in ASTM E 4740 (2004); viscometry, as described in J. Escobedo, et al., “Viscometric Determination of the Onset of Asphaltene Flocculation: A Novel Method,” Society of Petroleum Engineers, May 1995; optical microscopy; refractive indices measurement, as described in ASTM E 1218 (2012); vapor pressure osmometry, as described in U.S. Pat. No. 5,420,040 and Gawrey, et al., Instrumentation Science & Technology, 2004, 32(3), 247-253); gravimetric titrimetry, as described in U.S. Pat. No. 5,420,040; autoclaving; colloidal instability index, as described in Gawrey, et al., Instrumentation Science & Technology, 2004, 32(3), 247-253; detection of bubble points and asphaltene aggregation onset pressures by NIR, as described by Aske, et al., Energy & Fuels, 2002, 16, 1287-1295; nuclear magnetic resonance (NMR) relaxometry, as described in Prunelet et al., C R Chimie 7 (2004); pulsed-field gradient spin echo nuclear magnetic resonance (NMR), as described in Gawrey, et al., Instrumentation Science & Technology, 2004, 32(3), 247-253; small-angle neutron scattering, as described in Gawrey, et al., Instrumentation Science & Technology, 2004, 32(3), 247-253; saturates, asphaltenes, resins, aromatics (SARA) analysis, where A/R>0.35 is unstable, as described in Falkler et al., Hydrocarbon Processing, September 2010, 67-73; or any combination thereof.

As used herein, the term “solubility ratio” refers to a precipitation propensity of a crude oil determined by: (i) adding an asphaltene non-solvent to an initial volume of a crude oil; (ii) measuring the volume of the asphaltene non-solvent that causes asphaltene precipitation, e.g., by determining a minimum optical density as measured by near infrared spectroscopy; and (iii) dividing the volume of the asphaltene non-solvent added to the crude oil by the initial volume of the crude oil.

As used herein, the term “near-infrared spectrometry” or “NIR spectrometry” refers to spectroscopic methods that use the near-infrared region of the electromagnetic spectrum from about 800 nm to about 2,500 nm.

The methods discussed and described herein can be used to predict the compatibility of a wide range of different crude oils before blending, including operable proportions, to reduce, minimize, prevent, or eliminate asphaltene precipitation and/or one or more of the problems caused by asphaltene precipitation such as unplanned refinery events caused by asphaltene precipitation. The proportions of any number of crude oils in a blend and/or to be combined with one another to form a blend can also be determined to help optimize crude rate and/or crude blend compatibility. The methods and the associated calculations allow a precipitation propensity, such as a solubility ratio as measured by near-infrared spectrometry, to be determined for the crude oil blend components.

Accordingly, the method for selecting crude oils to prepare a crude oil blend can include independently measuring, estimating, or otherwise determining a precipitation propensity of a first crude oil and at least one second crude oil. The at least one second crude oil can include 1, 2, 3, 4, 5, 6, 7, 8, 9, 10, or more different crude oils. At least one of the first crude oil and the at least one second crude oil can include asphaltenes. The products of the precipitation propensity and volume percent proposed in the crude oil blend can be summed for each of the first crude oil and the second crude oil to provide a sum for the crude oil blend. The crude oil blend can be modified, e.g., the amount of the first crude oil and/or the amount of the second crude oil can be adjusted, if the sum is less than a predetermined cutoff.

In some examples, when the second crude oil does not include asphaltenes, the precipitation propensity for the second crude oil, when blended with the first crude oil, can be back calculated using a measured precipitation propensity of the crude oil blend and the precipitation propensity of the first crude oil sample. In one or more examples, modification of the crude oil blend can include replacing at least a portion of the second crude oil with a different crude oil; adjusting a volume percent of the second crude oil relative to a volume percent of the first crude oil; adding an additional crude oil, where the additional crude oil is different than the first crude oil and the second crude oil; excluding the second crude oil from the crude oil blend; adding at least one compatibility additive; or any combination thereof.

Suitable compatibility additives can reduce asphaltene flocculation or agglomeration in the crude oil blend. Illustrative compatibility agents can include, but are not limited to, diesel, kerosene, hexane-1-ol, toluene, organic polymers, biological oils, synthetic oils, alkylphenol-based resins, metal oxide-based colloidal hydrocarbon-based nanodispersions, or any mixture thereof. One exemplary compatibility additive can include MILESTONE® available from Baker Petrolite.

Methods for determining, measuring, estimating, or predicting the precipitation propensity of the composition can include, but are not limited to, volumetric solvent titrimetry with optical measurement, e.g., IR and/or NIR spectroscopy, including oil compatibility models and/or Asphaltene Stability Index (ASI) test using solvent-titration; solvent titrimetry with electrical measurement, e.g., conductivity and/or capacitance; solvent titrimetry with surface tension measurement; spot test; viscometry; optical microscopy; refractive indices measurement; vapor pressure osmometry; gravimetric titrimetry; autoclaving; colloidal instability index; detection of bubble points and asphaltene aggregation onset pressures by NIR; nuclear magnetic resonance (NMR) relaxometry; pulsed-field gradient spin echo nuclear magnetic resonance (NMR); small-angle neutron scattering; SARA analysis; or any combination thereof. In at least one specific example, the method for determining, measuring, estimating, or predicting the precipitation propensity can include volumetric solvent titrimetry with NIR optical measurement.

In at least one specific example, the precipitation propensity can be or include a solubility ratio, where the solubility ratio for each of the first crude oil sample and the at least one second crude oil sample can be determined by: adding to each of the first crude oil and the at least one second crude oil an asphaltene non-solvent. The volume of the asphaltene non-solvent that causes a minimum optical density, as measured by near infrared spectroscopy for each of the first crude oil and the second crude oil, can be measured. A solubility ratio for each of the first crude oil and the second crude oil can be calculated by dividing the volume of the asphaltene non-solvent added by the initial volume of the first crude oil or the at least one second crude oil. In certain examples, a number i can optionally be added to the solubility ratio, where the number i is a number large enough so that the solubility ratio is non-negative.

The temperature of the crude oil, e.g., the first crude oil and/or the second crude oil, can be maintained at a constant temperature or a substantially constant temperature when determining, measuring, estimating, or predicting the precipitation propensity. As used herein, the phrase “substantially constant temperature” refers to maintaining a temperature of the crude oil, when determining, measuring, estimating, or predicting the precipitation propensity of the crude oil, to within +/−5° C., +/−4° C., +/−3° C., +/−2° C., or +/−1° C., of a predetermined temperature. The predetermined temperature of the crude oil when the precipitation propensity is determined, measured, estimated, or predicted can be from a low of about 20° C., about 25° C., about 30° C., about 35° C., about 40° C., or about 45° C. to a high of about 50° C., about 55° C., about 60° C., about 65° C., about 70° C., about 75° C., about 80° C., or more. For example the crude oil can be at or within +/−5° C. of a temperature of 20° C., 25° C., 30° C., 35° C., 40° C., 45° C., 50° C., 55° C., 60° C., 65° C., 70° C., 75° C., or 80° C. when the precipitation propensity is determined, measured, estimated, or predicted.

The methods discussed and described herein can also include independently prescreening the at least one second crude oil to determine whether or not to include the second crude oil. The prescreening can include, but is not limited to, determining a measured precipitation propensity, such as a solubility ratio, of the crude oil blend; comparing the measured precipitation propensity with the calculated precipitation propensity or “sum” of the crude oil blend; and excluding the second crude oil if the measured precipitation propensity is greater than 20%, greater than 15%, greater than 10%, greater than 7%, or greater than 5% of the calculated precipitation propensity.

The methods discussed and described herein can be used in situations where not all of the components of the crude oil blends contain asphaltenes. In some examples, the first crude oil can include asphaltenes. For example, the first crude oil can include at least 0.17 wt % of asphaltenes, based on the weight of the first crude oil. In other examples, the at least one second crude oil can include asphaltenes. For example, the second crude oil can include at least 0.17 wt % of asphaltenes, based on the weight of the second crude oil. In yet other examples, each of the first crude oil and the second crude oil can independently contain or include asphaltenes. In still other examples, the first crude oil can include asphaltenes and the second crude oil can be free from asphaltenes or the second crude oil can include asphaltenes and the first crude oil can be free from asphaltenes. The asphaltenes can be independently present in the first crude oil and the second crude oil in an amount of at least 0.08%, at least 0.085%, at least 0.09%, at least 0.1%, at least 0.12%, at least 0.14%, at least 0.15%, at least 0.17 wt %, at least 0.2 wt %, at least 0.25 wt %, at least 0.3 wt %, at least 0.35 wt %, at least 0.4 wt %, at least 0.45 wt %, or at least 0.5 wt %, based on the weight of the first crude oil or the at least one second crude oil. The total amount of asphaltenes present in a blend that includes the first crude oil and the second crude oil can be at least 0.17 wt %, at least 0.2 wt %, at least 0.25 wt %, at least 0.3 wt %, at least 0.35 wt %, at least 0.4 wt %, at least 0.45 wt %, or at least 0.5 wt %, based on the combined weight of the first crude oil and the second crude oil.

A first asphaltene non-solvent can be added to an initial volume of a first crude oil sample representative of a first crude oil and a second asphaltene non-solvent can be added to an initial volume of at least one second crude oil sample representative of the second crude oil, where at least one of the first crude oil and the second crude oil includes asphaltenes. The first asphaltene non-solvent and the second asphaltene non-solvent can be the same or different with respect to one another. For example, the first asphaltene non-solvent can be the same as the second asphaltene non-solvent. A volume of the asphaltene non-solvent that causes a minimum optical density, as measured by near infrared spectroscopy, for each of the first crude oil sample and the second crude oil sample can be measured. The solubility ratio for first crude oil sample can be calculated by dividing the volume of the first asphaltene non-solvent added to the initial volume of the first crude oil sample by the initial volume of the first crude oil sample. The solubility ratio for the second crude oil sample can be calculated by dividing the volume of the second asphaltene non-solvent added to the initial volume of the second crude oil sample by the initial volume of the second crude oil sample.

The products of the solubility ratio and the volume percent proposed in the crude oil blend for each of the first crude oil sample and the at least one second crude oil sample can be added to provide a sum for proposed the crude oil blend. The composition of the proposed crude oil blend can be modified if the sum is less than a predetermined cutoff. The first crude oil and the second crude oil can be combined if the sum is equal to or greater than the predetermined cutoff.

In some examples, the method can also include adding a number i to the solubility ratio, where the number i is a number large enough so that the solubility ratio is non-negative. The number i can be a non-negative integer. For example, the number i can be equal to 1, 2, 3, 4, 5, or greater. In one example, if the solubility ratio is negative, the number i can be the smallest integer required to make the solubility ratio non-negative. The value for i, when added to the solubility ratio of the first crude oil and the second crude oil, can be the same or different with respect to one another.

The predetermined cutoff can be about 1.3, about 1.25, or about 1.2, when the asphaltene non-solvent is or includes n-heptane. For example, when the asphaltene non-solvent is or includes n-heptane, the predetermined cutoff can be about 1.1, about 1.13, about 1.15, about 1.17, about 1.2, or about 1.23 to about 1.27, about 1.3, about 1.33, about 1.35, about 1.37, or about 1.4.

The measured solubility ratio can be compared with the calculated precipitation propensity or “sum” of a proposed crude oil blend that includes the first crude oil and the at least one second crude oil. The amount of the second crude oil relative to the first crude oil can be adjusted or excluded from the proposed crude oil blend if the measured solubility ratio is greater than 20%, greater than 15%, or greater than 10% of the calculated precipitation propensity or sum of the crude oil blend.

The asphaltene non-solvent can be or include one or more normal paraffins. Illustrative normal paraffins can include, but are not limited to, n-pentane, n-hexane, n-heptane, n-octane, or any mixture thereof. For example, the asphaltene non-solvent can be or include n-pentane and/or n-heptane. In another example, the asphaltene non-solvent can be or include n-heptane.

A near infrared (NIR) spectrometer can be used as a detector to measure, determine, or otherwise estimate an asphaltene precipitation, agglomeration, and/or flocculation point of a crude oil sample. The asphaltene precipitation, agglomeration, and/or flocculation point occurs at the minimum optical density (OD). The minimum optical density can be measured at a wavelength from a low of about 800 nm, about 950 nm, about 1,100 nm, about 1,300 nm, about 1,400 nm, or about 1,500 nm to a high of about 1,600 nm, about 1,700 nm, about 1,800 nm, about 2,000 nm, about 2,200 nm, or about 2,500 nm. For example, the minimum optical density can be measured at a wavelength of about 1,400 nm, about 1,450 nm, about 1,500 nm, or about 1,550 nm to about 1,625 nm, about 1,650 nm, about 1,675 nm, about 1,700 nm, or about 1,725 nm. In another example, the minimum optical density can be measured at a wavelength of about 1,525 nm, about 1,550 nm, about 1,575 nm, about 1,600 nm, about 1,625 nm, about 1,650 nm, about 1,675 nm, or about 1,700 nm. It should be appreciated that the minimum optical density can be measured at any desired wavelength. In one embodiment, the wavelength can be at or about, e.g., +/−10 nm, a wavelength at which crude oil has a low absorbance. One particular wavelength crude oil has a low absorbance at and can be used to measure the minimum optical density can be at or about 1,600 nm.

As used herein, the term “optical density” refers to the attenuated measurement in the incident light due to absorbance and scattering by a medium through which the light travels. The optical density can be mathematically represented by the formula: OD=log (Iₒ/I), where “Iₒ” is the incident light and “I” is the transmitted light. Asphaltenes can flocculate from solution with the addition of an asphaltene non-solvent, e.g., an n-alkane such as n-heptane, and the OD can be affected by the flocculation of the asphaltenes. The optical density at a wavelength of about 1,600 nm can be of particular interest since it corresponds to a region associated with relatively low background absorbance for crude oil. As asphaltene non-solvent is added to a crude oil sample (also referred to as “titration”), the NIR absorbance and the OD at about 1,600 nm decrease initially due to dilution of the sample; however, asphaltene flocculation causes an increase in the OD as the transmitted light is reduced due to the scattering and absorbance of light by the flocculated asphaltenes. Hence, a minimum is observed in the OD at about 1,600 nm with addition of an asphaltene non-solvent such as n-heptane. It is the volume of asphaltene non-solvent corresponding to the minimum OD that is reported as the onset point of asphaltene flocculation and used in the solubility ratio.

### Examples

In order to provide a better understanding of the foregoing discussion, the following non-limiting examples are offered. All parts and percentages are by volume, unless otherwise stated. It should be understood that these examples are given by way of illustration only and are not to be construed as limiting in any manner.

The precipitation propensity of several heavy crude oils and blends thereof were measured. More particularly, the precipitation propensity of six (non-U.S.) heavy crude oils (crude oils A, B, C, D, E, and F) and blends made therefrom were measured. Crude oils A and C were blended together (51.75 vol % of crude oil A and 48.25 vol % of crude oil C) to prepare a base oil that was then blended with crude oils B, D, E, and F in different amounts. The amount of each crude oil blended together to prepare the various crude oil blends are shown in the Table below.

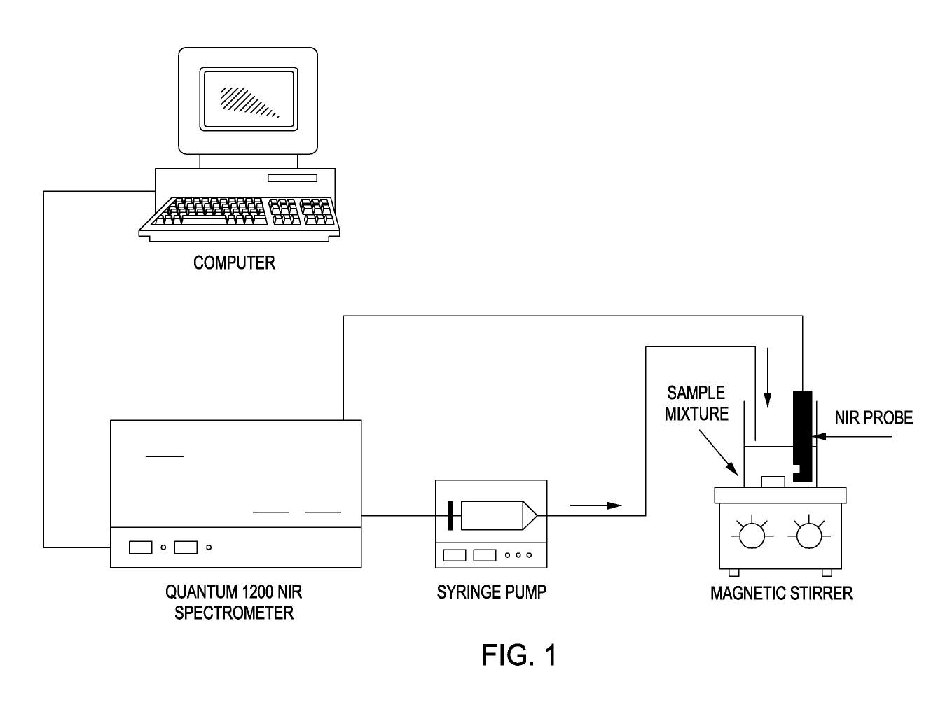

The amount of n-heptane required to initiate asphaltene precipitation for each crude oil or crude oil blend was measured using a NIR spectrometer (LT Industries, Inc. 1200 Plus Quantum fitted with a fiber optic sampling probe with a sapphire lens for transflectance measurements). The spectrometer had a band width of 5 nm. The probe was immersed in the oil with an effective path length of 6 mm. Titrations were carried out at a flow rate of 1 mL/min. The flow rate was controlled using a syringe pump. FIG. 1 depicts a schematic of the instrument set-up, which included a 1200 Plus Quantum NIR spectrometer that was used to measure the amount of n-heptane required to initiate asphaltene precipitation in the examples. The wavelength used to measure the crude oils and blends thereof was about 1,600 nm, but an NIR spectra over a spectral range of about 1,200 nm to about 2,400 nm could have been used.

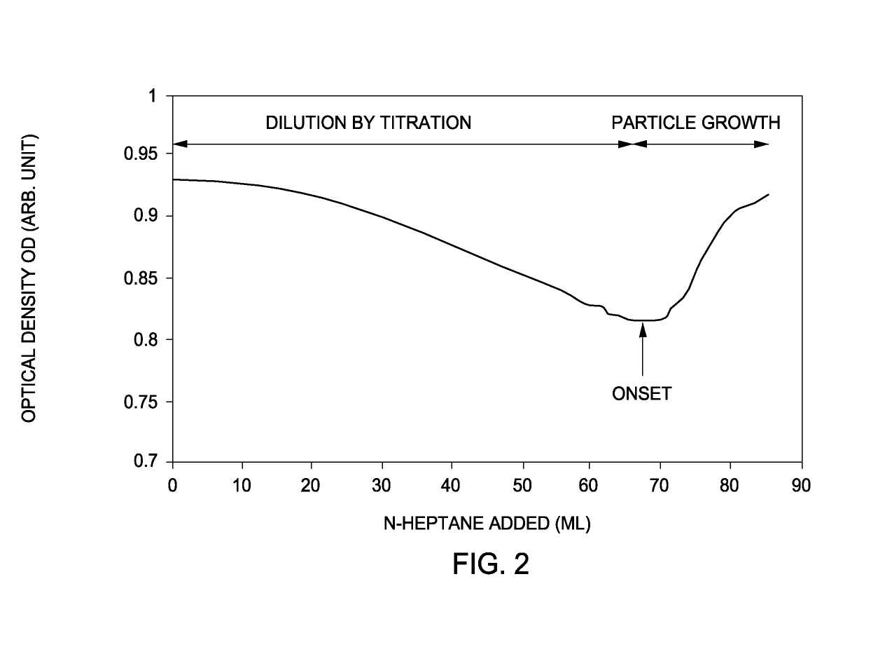

To measure the amount of n-heptane required to initiate the asphaltene precipitation, about 35 mL of the crude oil was pipetted into a titration cup (Note: Samples were prepared at time of use). The stir plate was set to about 140 rpm and the pump was set to about 1 mL/min. The pump was filled with n-heptane titrant and the air was purged out of the tubing so that there was a continuous flow of titrant. A stir bar was put into the vessel and placed on the stir plate and the probe and titrant tube were lowered into the titration cup. The titration was started and the crude oil sample was titrated and the spectrum at 1,600 nm was observed. For illustrative purposes, a graphical depiction of the optical density as a function of the volume of n-heptane during titration of an exemplary crude oil, measured using near infrared spectrometry (1,600 nm), is shown in FIG. 2. The total amount of n-heptane required to titrate the crude oil (lowest point of the curve in FIG. 2) was determined by the inflection in the spectra curve. The titrations for the examples were carried out at room temperature, e.g., about 25° C.+/−5° C. It should be noted that the titrations could have been carried out at an elevated temperature, e.g., about 50° C.+/−5° C., which could improve the measurement for low asphaltene containing crude oils.

The product of the solubility ratio and volume percent proposed in the crude oil blend for each of the first crude oil sample and the at least one second crude oil sample were summed to calculate a sum for the crude oil blend. This blending method accounted for crude oils that had excess solubility. When blending light crude oil, such as domestic (U.S.) crude oils, due to the lack of asphaltenes it was found that these ratios could be negative. To solve this problem, a value of 1 (one) was added to the calculation to make the solubility ratio a positive number. Any non-negative integer can work. However, low non-negative integers, such as one, can be preferred to keep the solubility ratio numbers smaller and, therefore, easier to work with.

The NIR ratio was calculated according to the following equation:

$$Solubility Ratio = [ \frac{Volume of Asphaltene Non \text{-} Solvent Added ( mL )}{Initial Volume of Crude Oil Sample ( mL )} ] + i ,$$

where i was equal to 1.

Blending the crude oils by using the NIR ratio took into account the crude oils that had excess solubility (excess resins), which allowed the insolubility to blend down. This allowed crude oil with unstable asphaltenes to be blended with a crude oil that contained excess resins or excess solubility to produce a more stable crude oil blend.

The solubility and NIR ratios were then compared to the predicted NIR ratio that was calculated by blending the crude oil NIR ratios by volume. The difference between the actual (measured) and calculated (predicted) NIR ratios are also shown in the Table below. The % Difference was determined by multiplying the difference between the actual solubility and the predicted solubility by 100 and dividing that value by the average of the Actual and Predicted NIR ratios. The % Difference is represented by the formula:

$$\% Difference = [ \frac{ \begin{matrix}Difference between the Actual \\ and the Predicted NIR Ratios \times 100\end{matrix}}{Average of the Actual and Predicted NIR Ratios} ]$$

TABLE 2

| | NIR | | Difference | |
|---|---|---|---|---|
| Crude Blends | Ratio | NIR Ratio | of Actual vs. | % |
| (volume %) | (Actual) | (Predicted) | Predicted | Difference |
| A | 2.11 | | | |
| C | 3.04 | | | |
| Base | 2.52 | 2.56 | 0.04 | 1.57 |
| [51.75/48.25 (A/C)] | | | | |
| 70.83/29.17 (A/C) | 2.40 | 2.38 | 0.02 | 0.3 |
| 53.02/46.98 (A/C) | 2.64 | 2.55 | 0.09 | 3.47 |
| 26.67/73.33 (A/C) | 2.76 | 2.79 | 0.03 | 1.08 |
| B | 6.20 | | | |
| 89.99/10.01 (A/B) | 2.40 | 2.52 | 0.12 | 4.88 |
| 74.59/25.41 (A/B) | 2.90 | 3.15 | 0.25 | 8.26 |
| 50.65/49.35 (A/B) | 3.80 | 4.13 | 0.33 | 8.32 |
| 89.34/10.66 (Base/B) | 2.90 | 2.95 | 0.05 | 1.71 |
| 52.56/47.44 (Base/B) | 3.93 | 4.29 | 0.35 | 8.52 |
| 51.29/48.71 (Base/B) | 4.00 | 4.33 | 0.33 | 7.92 |
| E | 2.84 | | | |
| 50.77/49.23 (E/Base) | 2.56 | 2.70 | 0.14 | 3.36 |
| 24.26/75.74 (E/Base) | 2.48 | 2.63 | 0.15 | 5.87 |
| 12.26/87.74 (E/Base) | 2.40 | 2.59 | 0.19 | 7.62 |
| D | 1.86 | | | |
| 75.09/24.91 (D/Base) | 1.97 | 2.03 | 0.06 | 2.83 |
| 50.04/49.96 (D/Base) | 2.03 | 2.21 | 0.18 | 8.49 |
| 26.52/73.48 (D/Base) | 2.18 | 2.37 | 0.20 | 8.79 |
| F | 2.60 | | | |
| 53.13/46.87 (F/Base) | 2.52 | 2.58 | 0.06 | 2.35 |
| 26.29/73.71 (F/Base) | 2.44 | 2.57 | 0.13 | 5.19 |

A crude oil that cannot sufficiently solubilize asphaltenes will generally tend to flocculate the asphaltenes out of solution. This may occur in tanks, crude oil de-salters, crude oil pre-heat trains, and/or crude heaters, for example. Asphaltenes not in solution can cause coking in the heat exchangers/crude heaters and/or can also be rejected in the crude de-salter as organic solids potentially causing downstream waste water treatment problems. The methods discussed and described herein can be used to determine if the crude oil being charged to the crude unit is stable and not precipitating asphaltenes and/or help in optimizing the amount of crude oil processed while staying within compatibility limits.

Embodiments of the present disclosure further relate to any one or more of the following paragraphs:

1. A method for selecting crude oils to prepare a crude oil blend, comprising: providing a first crude oil sample representative of a first crude oil; providing at least one second crude oil sample representative of at least one second crude oil; wherein at least one of the first crude oil and the at least one second crude oil comprises asphaltenes; independently measuring a precipitation propensity of the first crude oil sample and the at least one second crude oil sample; summing the products of the precipitation propensity and volume percent proposed in the crude oil blend for each of the first crude oil sample and the at least one second crude oil sample to calculate a sum for the crude oil blend; and modifying the crude oil blend if the sum is less than a predetermined cutoff.

2. The method according to paragraph 1, wherein the at least one second crude oil sample does not comprise asphaltenes, and wherein the precipitation propensity for the at least one second crude oil sample, when blended with the first crude oil sample, is back calculated using a measured precipitation propensity of the crude oil blend and the precipitation propensity of the first crude oil sample.

3. The method according to paragraph 1 or 2, wherein the precipitation propensity is measured using a technique comprising: volumetric solvent titrimetry with optical measurement; volumetric solvent titrimetry with electrical measurement; volumetric solvent titrimetry with surface tension measurement; spot test; viscometry; optical microscopy; refractive indices measurement; vapor pressure osmometry; gravimetric titrimetry; autoclaving; colloidal instability index; detection of bubble points and asphaltene aggregation onset pressure; nuclear magnetic resonance (NMR) relaxometry; pulsed-field gradient spin echo nuclear magnetic resonance (NMR); small-angle neutron scattering; SARA analysis; or any combination thereof.

4. The method according to any one of claims 1 to 3, wherein the precipitation propensity is measured using volumetric solvent titrimetry with near infrared (NIR) optical measurement.

5. The method according to any one of paragraphs 1 to 4, wherein the precipitation propensity is a solubility ratio, wherein the solubility ratio for each of the first crude oil sample and the at least one second crude oil sample is determined by: adding to each of the first crude oil sample and the at least one second crude oil sample an asphaltene non-solvent; measuring the volume of the asphaltene non-solvent that causes a minimum optical density as measured by near infrared spectroscopy for each of the first crude oil sample and the at least one second crude oil sample; and calculating a solubility ratio for each of the first crude oil sample and the at least one second crude oil sample by dividing the volume of the asphaltene non-solvent added by the initial volume of the first crude oil sample or the at least one second crude oil sample.

6. The method according to paragraph 5, wherein a number i is added to the solubility ratio, and wherein the number i is a number large enough so that the solubility ratio is non-negative.

7. The method according to paragraph 5 or 6, wherein the asphaltene non-solvent comprises at least one normal paraffin.

8. The method according to any one of paragraphs 5 to 7, wherein the asphaltene non-solvent comprises n-pentane, n-hexane, n-heptane, n-octane, or any mixture thereof.

9. The method according to any one of paragraphs 5 to 8, wherein the asphaltene non-solvent comprises n-heptane.

10. The method according to any one of paragraphs 5 to 9, wherein the minimum optical density is measured at a wavelength of about 1,400 nm to about 1,725 nm.

11. The method according to any one of paragraphs 5 to 10, wherein the minimum optical density is measured at a wavelength of about 1,600 nm.

12. The method according to any one of paragraphs 1 to 11, wherein modifying the crude oil blend comprises: replacing at least a portion of the at least one second crude oil with a different crude oil; adjusting the volume percent of the at least one second crude oil relative to the volume percent of the first crude oil; adding an additional crude oil; wherein the additional crude oil is different than the first crude oil and the at least one second crude oil; excluding the at least one second crude oil from some the crude oil blend adding at least one compatibility additive; or any combination thereof.

13. The method according to any one of paragraphs 1 to 12, further comprising independently prescreening each of the at least one second crude oil samples to determine whether or not to include the at least one second crude oil.

14. The method according to paragraph 13, wherein the prescreening comprises: determining a measured precipitation propensity of the crude oil blend; comparing the measured precipitation propensity with the sum of the crude oil blend; and excluding the at least one second crude oil sample if the calculated precipitation propensity differs by more than about 20% from the sum of the crude oil blend.

15. The method according to paragraph 13, wherein the prescreening comprises: determining a measured precipitation propensity of the crude oil blend; comparing the measured precipitation propensity with the sum of the crude oil blend; and excluding the at least one second crude oil sample if the calculated precipitation propensity differs by more than about 10% from the sum of the crude oil blend.

16. The method according to any one of paragraphs 1 to 15, wherein the first crude oil comprises an asphaltene.

17. The method according to paragraph 16, wherein the asphaltene is present at a level of at least about 0.17 wt %, based on the weight of the first crude oil.

18. The method according to any one of paragraphs 1 to 17, wherein the at least one second crude oil comprises an asphaltene.

19. The method according to paragraph 18, wherein the asphaltene is present at a level of at least about 0.17 wt %, based on the weight of the at least second crude oil.

20. The method according to any one of paragraphs 1 to 19, wherein both of the first crude oil and the at least one second crude oil each comprise an asphaltene.

21. The method according to paragraph 20, wherein the asphaltene is present, independently, in the first crude oil and the at least one second crude oil, at a level of at least about 0.5 wt %, based on the weight of the first crude oil or the at least one second crude oil.

22. A method for selecting crude oils to prepare a crude oil blend, comprising: providing an initial volume of a first crude oil sample representative of a first crude oil; providing an initial volume of at least one second crude oil sample representative of at least one second crude oil, wherein at least one of the first crude oil and the at least one second crude oil comprises an asphaltene; adding to each of the first crude oil sample and the at least one second crude oil sample an asphaltene non-solvent; measuring the volume of the asphaltene non-solvent that causes a minimum optical density as measured by near infrared spectroscopy for each of the first crude oil sample and the at least one second crude oil sample; calculating a solubility ratio for each of the first crude oil sample and the at least one second crude oil sample by dividing the volume of the asphaltene non-solvent added by the initial volume of the first crude oil sample or the at least one second crude oil sample; summing the products of the solubility ratio and volume percent proposed in the crude oil blend for each of the first crude oil sample and the at least one second crude oil sample to calculate a sum for the crude oil blend; and modifying the crude oil blend if the sum is less than a predetermined cutoff.

23. The method according to paragraph 22, further comprising adding a number i to the solubility ratio, wherein the number i is a number large enough so that the solubility ratio is non-negative.

24. The method according to paragraph 23, wherein i is 1.

25. The method according to paragraph 24, wherein the predetermined cutoff is about 1.3 when the asphaltene non-solvent comprises n-heptane.

26. The method according to paragraph 24, wherein the predetermined cutoff is about 1.25, when the asphaltene non-solvent comprises n-heptane.

27. The method according to paragraph 24, wherein the predetermined cutoff is about 1.2, when the asphaltene non-solvent comprises n-heptane.

28. The method according to any one of paragraphs 22 to 27, wherein modifying the crude oil blend comprises: replacing at least a portion of the at least one second crude oil with a different crude oil; adjusting the volume percent of the at least one second crude oil relative to the volume percent of the first crude oil; adding an additional crude oil; wherein the additional crude oil is different than the first crude oil and the at least one second crude oil; excluding the at least one second crude oil from some the crude oil blend; adding at least one compatibility additive; or any combinations thereof.

29. The method according to any one of paragraphs 22 to 28, further comprising independently prescreening each of the at least one second crude oil samples to determine whether or not to include the at least one second crude oil samples.

30. The method according to paragraph 29, wherein the prescreening comprises: determining a measured solubility ratio of the crude oil blend; comparing the measured solubility ratio with the sum of the crude oil blend; and excluding the at least one second crude oil sample if the calculated solubility ratio differs by more than about 20% from the sum of the crude oil blend.

31. The method according to paragraph 29, wherein the prescreening comprises: determining a measured solubility ratio of the crude oil blend; comparing the measured solubility ratio with the sum of the crude oil blend; and excluding the at least one second crude oil sample if the calculated solubility ratio differs by more than about 10% from the sum of the crude oil blend.

32. The method according to any one of paragraphs 22 to 31, wherein the first crude oil comprises an asphaltene.

33. The method according to paragraph 32, wherein the asphaltene is present at a level of at least 0.17 wt %, based on the weight of the first crude oil.

34. The method according to any one of paragraphs 22 to 33, wherein the at least one second crude oil comprises an asphaltene.

35. The method according to paragraph 34, wherein the asphaltene is present at a level of at least 0.17 wt %, based on the weight of the at least second crude oil.

36. The method according to any one of paragraphs 22 to 35, wherein both of the first crude oil and the at least one second crude oil each independently comprise an asphaltene.

37. The method according to paragraph 36, wherein the asphaltene is present, independently, in the first crude oil and the at least one second crude oil, at a level of at least 0.5 wt %, based on the weight of the first crude oil or the at least one second crude oil.

38. The method according to any one of paragraphs 22 to 37, wherein the asphaltene non-solvent comprises at least one normal paraffin.

39. The method according to paragraph 38, wherein the at least one normal paraffin comprises n-pentane, n-hexane, n-heptane, n-octane, or any mixture thereof.

40. The method according to paragraph 38, wherein the at least one normal paraffin comprises n-heptane.

41. The method according to any one of paragraphs 22 to 40, wherein the minimum optical density is measured at a wavelength of about 1,400 nm to about 1,725 nm.

42. The method according to any one of paragraphs 22 to 40, wherein the minimum optical density is measured at a wavelength of about 1,600 nm.

43. The method according to any one of paragraphs 22 to 40, wherein the minimum optical density is measured at a wavelength of about 1,550 nm to about 1,650 nm.

44. A method for selecting crude oils for blending with one another, comprising: independently measuring a precipitation propensity of a first crude oil and at least one second crude oil, wherein at least one of the first crude oil and the at least one second crude oil comprises asphaltenes; adding a product of the precipitation propensity and a volume percent for each of the first crude oil and the at least one second crude oil in a proposed crude oil blend to provide a sum for the crude oil blend; modifying the proposed crude oil blend if the sum is less than a predetermined cutoff; and blending the first crude oil and the second crude oil with one another when the sum is greater than or equal to the predetermined cutoff value.

45. The method according to paragraph 44, wherein modifying the crude oil blend comprises: replacing a portion of the at least one second crude oil with a different crude oil; adjusting the volume percent of the at least one second crude oil relative to the volume percent of the first crude oil; adding an additional crude oil; wherein the additional crude oil is different than the first crude oil and the at least one second crude oil; adding at least one compatibility additive; or any combination thereof.

46. The method according to paragraph 44 or 45, wherein the precipitation propensity is measured using a technique comprising: volumetric solvent titrimetry with optical measurement; volumetric solvent titrimetry with electrical measurement; volumetric solvent titrimetry with surface tension measurement; spot test; viscometry; optical microscopy; refractive indices measurement; vapor pressure osmometry; gravimetric titrimetry; autoclaving; colloidal instability index; detection of bubble points and asphaltene aggregation onset pressure; nuclear magnetic resonance (NMR) relaxometry; pulsed-field gradient spin echo nuclear magnetic resonance (NMR); small-angle neutron scattering; SARA analysis; or any combination thereof.

47. The method according to any one of paragraphs 44 to 46, wherein the precipitation propensity is measured using volumetric solvent titrimetry with NIR optical measurement.

48. The method according to any one of paragraphs 44 to 47, wherein the precipitation propensity is a solubility ratio.

49. The method according to paragraph 48, wherein the solubility ratio for each of the first crude oil sample and the at least one second crude oil sample is determined by: adding to each of the first crude oil sample and the at least one second crude oil sample an asphaltene non-solvent; measuring the volume of the asphaltene non-solvent that causes a minimum optical density as measured by near infrared spectroscopy for each of the first crude oil sample and the at least one second crude oil sample; and calculating a solubility ratio for each of the first crude oil sample and the at least one second crude oil sample by dividing the volume of the asphaltene non-solvent added by the initial volume of the first crude oil sample or the at least one second crude oil sample.

50. The method according to paragraph 49, wherein a number i is added to the solubility ratio, and wherein the number i is a number large enough so that the solubility ratio is non-negative.

51. The method according to paragraphs 49 or 50, wherein the asphaltene non-solvent comprises at least one normal paraffin.

52. The method according to any one of paragraphs 49 to 52, wherein the one normal paraffin comprises n-pentane, n-hexane, n-heptane, n-octane, or any mixture thereof.

53. The method according to any one of paragraphs 49 to 53, wherein the one normal paraffin comprises n-heptane.

54. The method according to any one of paragraphs 49 to 54, wherein the minimum optical density is measured at a wavelength of about 1,400 nm to about 1,725 nm.

55. The method according to any one of paragraphs 49 to 55, wherein the minimum optical density is measured at a wavelength of about 1,600 nm.

56. A method for selecting crude oils for blending with one another, comprising: measuring a precipitation propensity of a first crude oil and at least one second crude oil, wherein at least one of the first crude oil and the at least one second crude oil comprise asphaltenes; multiplying the precipitation propensity of the first crude oil with a proposed volume percent of the first crude oil in the crude oil blend to provide a first product; multiplying the precipitation propensity of the second crude oil with a proposed volume percent of the second crude oil in the crude oil blend to provide a second product; adding the first product and the second product to provide a sum for the crude oil blend; and blending the first crude oil and the second crude with one another if the sum is greater than a predetermined cutoff; or modifying the proposed crude oil blend if the sum is less than the predetermined cutoff such that the sum is increased to the predetermined cutoff or is greater than the predetermined cutoff.

57. A crude oil blend, comprising: a first crude oil and at least one second crude oil, wherein at least one of the first crude oil and the second crude oil comprise asphaltenes when combined with one another to produce the crude oil blend, wherein a calculated precipitation propensity of the crude oil blend is confirmed to be greater than a predetermined cutoff prior to combining the first crude oil and the second crude oil with one another to produce the crude oil blend, wherein a calculation of the precipitation propensity of the crude oil comprises: independently measuring a precipitation propensity of the first crude oil and the at least one second crude oil; and adding a product of the precipitation propensity and a volume percent for each of the first crude oil and the at least one second crude oil in the crude oil blend.

58. The method or blend according to paragraphs 56 or 57, wherein the calculated precipitation propensity is a solubility ratio, wherein the solubility ratio for each of the first crude oil and the at least one second crude oil is determined by: adding to each of the first crude oil and the at least one second crude oil an asphaltene non-solvent; measuring the volume of the asphaltene non-solvent that causes a minimum optical density as measured by near infrared spectroscopy for each of the first crude oil and the at least one second crude oil; and calculating a solubility ratio for each of the first crude oil and the at least one second crude oil by dividing the volume of the asphaltene non-solvent added to the first crude oil or the at least one second crude oil by the initial volume of the first crude oil or the at least one second crude oil, respectively.

59. The method or blend according to paragraph 58, wherein a number i is added to the solubility ratio, and wherein the number i is a number large enough so that the solubility ratio is non-negative.

60. The method or blend according to paragraph 58 or 59, wherein the asphaltene non-solvent comprises at least one normal paraffin.

61. The method or blend according to any one of paragraphs 58 to 60, wherein the one normal paraffin comprises n-pentane, n-hexane, n-heptane, n-octane, or any mixture thereof.

62. The method or blend according to any one of paragraphs 58 to 61, wherein the one normal paraffin comprises n-heptane.

63. The method or blend according to any one of paragraphs 58 to 62, wherein the minimum optical density is measured at a wavelength of about 1,400 nm to about 1,725 nm.

64. The method or blend according to any one of paragraphs 58 to 63, wherein the minimum optical density is measured at a wavelength of about 1,600 nm.

65. A method for selecting crude oils to prepare a crude oil blend, comprising: measuring a precipitation propensity of a first crude oil; measuring a precipitation propensity of at least one second crude oil, wherein at least one of the first crude oil and the second crude oil comprises asphaltenes; multiplying the precipitation propensity of the first crude oil by a proposed volume percent of the first crude oil in a proposed crude oil blend to provide a first value; multiplying the precipitation propensity of the second crude oil by a proposed volume percent of the second crude oil in the proposed crude oil blend to provide a second value; modifying a composition of the proposed crude oil blend if a sum of the first value and the second value is less than a predetermined cutoff.

66. The method according to paragraph 65, wherein: the first crude oil and the second crude oil are combined with one another to produce the proposed crude oil blend, the second crude oil does not comprise asphaltenes, and the precipitation propensity for the second crude oil, when blended with the first crude oil, is back calculated using a measured precipitation propensity of the proposed crude oil blend and the precipitation propensity of the first crude oil.

67. The method according to any one of paragraphs 65 to 66, wherein measuring the precipitation propensities of the first crude oil and the second crude oil comprises: volumetric solvent titrimetry with optical measurement; volumetric solvent titrimetry with electrical measurement; volumetric solvent titrimetry with surface tension measurement; spot testing; viscometry; optical microscopy; refractive indices measurement; vapor pressure osmometry; gravimetric titrimetry; autoclaving; colloidal instability index; detection of bubble points and asphaltene aggregation onset pressure; nuclear magnetic resonance (NMR) relaxometry; pulsed-field gradient spin echo nuclear magnetic resonance (NMR); small-angle neutron scattering; saturates, asphaltenes, resins, aromatics (SARA) analysis; or any combination thereof.

68. The method according to any one of paragraphs 65 to 67, wherein the precipitation propensities of the first crude oil and the precipitation propensity of the second crude oil are measured by volumetric solvent titrimetry with near infrared optical measurement.

69. The method according to any one of paragraphs 65 to 68, wherein the precipitation propensities of the first crude oil and the second crude oil are solubility ratios, wherein the solubility ratio for each of the first crude oil and the second crude oil is determined by: adding an asphaltene non-solvent to each of an initial volume of the first crude oil and an initial volume of the second crude oil; measuring a volume of the asphaltene non-solvent that causes a minimum optical density as measured by near infrared spectroscopy in each of the first crude oil and the second crude oil; calculating a solubility ratio of the first crude oil by dividing the volume of the asphaltene non-solvent that causes the minimum optical density in the first crude oil by the initial volume of the first crude oil; and calculating a solubility ratio of the second crude oil by dividing the volume of the asphaltene non-solvent that causes the minimum optical density in the second crude oil by the initial volume of the second crude oil.

70. The method according to paragraph 69, wherein a number i is added to the solubility ratio of the first crude oil and to the solubility ratio of the second crude oil, and wherein the number i is a number large enough to make the solubility ratio of the first crude oil and the solubility of the second crude oil both non-negative.

71. The method according to paragraph 69 or 70, wherein the asphaltene non-solvent comprises a normal paraffin.

72. The method according to any one of paragraphs 69 to 71, wherein the asphaltene non-solvent comprises n-pentane, n-hexane, n-heptane, n-octane, or any mixture thereof.

73. The method according to any one of paragraphs 69 to 72, wherein the asphaltene non-solvent is n-pentane, and wherein the predetermined cutoff is 1.2.

74. The method according to any one of paragraphs 69 to 73, wherein the minimum optical density is measured at a wavelength of about 1,400 nm to about 1,725 nm.

75. The method according to any one of paragraphs 65 to 74, wherein the sum of the first value and the second value is less than the predetermined cutoff, and wherein modifying the composition of the proposed crude oil blend comprises: replacing at least a portion of the second crude oil with a different crude oil; adjusting a volume percent of the second crude oil relative to a volume percent of the first crude oil; adding an additional crude oil that is different than the first crude oil and the second crude oil; adding a compatibility additive to the crude oil blend; or any combination thereof.

76. The method according to any one of paragraphs 65 to 75, further comprising prescreening the second crude oil to determine whether or not to include the second crude oil, wherein the prescreening comprises: combining the first crude oil and the second crude oil to produce the proposed crude oil blend; measuring a precipitation propensity of the proposed crude oil blend; comparing the measured precipitation propensity of the proposed crude oil blend to the sum of the first value and the second value; and excluding the second crude oil if the measured precipitation propensity of the proposed crude oil blend differs from the sum of the first value and the second value by more than 20%.

77. The method according to any one of paragraphs 65 to 76, wherein of the first crude oil and the second crude oil comprises at least 0.17 wt % of asphaltenes.

78. A method for selecting crude oils for blending with one another, comprising: proposing an amount of a first crude oil and an amount of at least one second crude oil for combining with one another to produce a proposed crude oil blend, wherein at least one of the first crude oil and the second crude oil comprise asphaltenes; measuring a precipitation propensity of the first crude oil and the second crude oil; multiplying the precipitation propensity of the first crude oil by a volume percent of the first crude oil in the proposed crude oil blend to provide a first value; multiplying the precipitation propensity of the second crude oil by a volume percent of the second crude oil in the proposed crude oil blend to provide a second value; adding the first value and the second value to provide a sum for the proposed crude oil blend; and (1) combining the first crude oil and the second crude with one another to produce the proposed crude oil blend if the sum for the proposed crude oil blend is equal to or greater than a predetermined cutoff; or (2) modifying a composition of the proposed crude oil blend if the sum for the proposed crude oil blend is less than the predetermined cutoff to produce a modified proposed crude oil blend, wherein a sum for the modified proposed crude oil blend is equal to or greater than the predetermined cutoff.

79. The method according to paragraph 78, wherein the precipitation propensity of the first crude oil and the precipitation propensity of the second crude oil are solubility ratios, wherein the solubility ratio for each of the first crude oil and the second crude oil sample is determined by: adding an asphaltene non-solvent to a volume of the first crude oil and a volume of the second crude oil; measuring a volume of the asphaltene non-solvent that causes a minimum optical density by near infrared spectroscopy for each of the volume of the first crude oil and the volume of the second crude oil; dividing the volume of the asphaltene non-solvent that causes the minimum optical density in the first crude oil by the volume of the first crude oil to provide the solubility ratio of the first crude oil; and dividing the volume of the asphaltene non-solvent that causes the minimum optical density in the second crude oil by the volume of the second crude oil to provide the solubility ratio of the second crude oil.

80. The method according to paragraph 78 or 79, wherein: a number i is added to the solubility ratio of the first crude oil and to the solubility ratio of the second crude oil, the number i is a number large enough to make the solubility ratio of the first crude oil and the solubility of the second crude oil both non-negative, the asphaltene non-solvent comprises n-pentane, and the predetermined cutoff is 1.2.

81. The method according to any one of paragraphs 78 to 80, wherein: the first crude oil and the second crude oil are combined with one another to produce the proposed crude oil blend, the second crude oil does not comprise asphaltenes, and the precipitation propensity for the second crude oil, when blended with the first crude oil, is back calculated using a measured precipitation propensity of the proposed crude oil blend and the precipitation propensity of the first crude oil.

82. A method for selecting crude oils for blending with one another, comprising: proposing an amount of a first crude oil and an amount of at least one second crude oil for combining with one another to produce a proposed crude oil blend, wherein at least one of the first crude oil and the second crude oil comprise asphaltenes; measuring a solubility ratio of the first crude oil and a solubility ratio of the second crude oil, wherein the solubility ratio for each of the first crude oil and the second crude oil is measured by: adding to a volume of each of the first crude oil and the second crude oil a normal paraffin; measuring a volume of the normal paraffin that causes a minimum optical density by near infrared spectroscopy for each of the first crude oil and the second crude oil; dividing the volume of the asphaltene non-solvent that causes the minimum optical density in the first crude oil by the volume of the first crude oil to provide the solubility ratio of the first crude oil; and dividing the volume of the asphaltene non-solvent that causes the minimum optical density in the second crude oil by the volume of the second crude oil to provide the solubility ratio of the second crude oil; multiplying the solubility ratio of the first crude oil by a volume percent of the first crude oil in the proposed crude oil blend to provide a first value; multiplying the solubility ratio of the second crude oil by a volume percent of the second crude oil in the proposed crude oil blend to provide a second value; adding the first value and the second value to provide a sum for the proposed crude oil blend; and (1) combining the first crude oil and the second crude oil with one another to produce the proposed crude oil blend if the sum for the proposed crude oil blend is equal to or greater than a predetermined cutoff; or (2) modifying a composition of the proposed crude oil blend if the sum for the proposed crude oil blend is less than the predetermined cutoff to produce a modified proposed crude oil blend, wherein a sum for the modified proposed crude oil blend is equal to or greater than the predetermined cutoff.

83. The method according to paragraph 82, at least one of the first crude oil and the second crude oil comprises at least 0.17 wt % of asphaltenes, the one normal paraffin comprises n-pentane, n-heptane, or a mixture thereof, the minimum optical density is measured at a wavelength of about 1,550 nm to about 1,650 nm, the composition of the proposed crude oil blend is modified to produce the modified proposed crude oil blend, and modifying the composition of the crude oil blend comprises: replacing at least a portion of the second crude oil with a different crude oil; adjusting a volume percent of the second crude oil relative to a volume percent of the first crude oil; adding an additional crude oil that is different than the first crude oil and the second crude oil; adding a compatibility additive to the crude oil blend; or any combination thereof.

84. The method according to paragraph 82 or 83, wherein: the first crude oil and the second crude oil are combined with one another to produce the proposed crude oil blend, the second crude oil does not comprise asphaltenes, and the precipitation propensity for the second crude oil, when blended with the first crude oil, is back calculated using a measured precipitation propensity of the proposed crude oil blend and the precipitation propensity of the first crude oil.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

1. A method for selecting crude oils to prepare a crude oil blend, comprising:
measuring a precipitation propensity of a first crude oil;
measuring a precipitation propensity of at least one second crude oil, wherein at least one of the first crude oil and the second crude oil comprises asphaltenes;
multiplying the precipitation propensity of the first crude oil by a proposed volume percent of the first crude oil in a proposed crude oil blend to provide a first value;
multiplying the precipitation propensity of the second crude oil by a proposed volume percent of the second crude oil in the proposed crude oil blend to provide a second value;
modifying a composition of the proposed crude oil blend if a sum of the first value and the second value is less than a predetermined cutoff.

2. The method of claim 1, wherein:
the first crude oil and the second crude oil are combined with one another to produce the proposed crude oil blend,
the second crude oil does not comprise asphaltenes, and
the precipitation propensity for the second crude oil, when blended with the first crude oil, is back calculated using a measured precipitation propensity of the proposed crude oil blend and the precipitation propensity of the first crude oil.

3. The method of claim 1, wherein measuring the precipitation propensities of the first crude oil and the second crude oil comprises: volumetric solvent titrimetry with optical measurement; volumetric solvent titrimetry with electrical measurement; volumetric solvent titrimetry with surface tension measurement; spot testing; viscometry; optical microscopy; refractive indices measurement; vapor pressure osmometry; gravimetric titrimetry; autoclaving; colloidal instability index; detection of bubble points and asphaltene aggregation onset pressure; nuclear magnetic resonance (NMR) relaxometry; pulsed-field gradient spin echo nuclear magnetic resonance (NMR); small-angle neutron scattering; saturates, asphaltenes, resins, aromatics (SARA) analysis; or any combination thereof.

4. The method of claim 1, wherein the precipitation propensities of the first crude oil and the precipitation propensity of the second crude oil are measured by volumetric solvent titrimetry with near infrared optical measurement.

5. The method of claim 1, wherein the precipitation propensities of the first crude oil and the second crude oil are solubility ratios, wherein the solubility ratio for each of the first crude oil and the second crude oil is determined by:
adding an asphaltene non-solvent to each of an initial volume of the first crude oil and an initial volume of the second crude oil;
measuring a volume of the asphaltene non-solvent that causes a minimum optical density as measured by near infrared spectroscopy in each of the first crude oil and the second crude oil;
calculating a solubility ratio of the first crude oil by dividing the volume of the asphaltene non-solvent that causes the minimum optical density in the first crude oil by the initial volume of the first crude oil; and
calculating a solubility ratio of the second crude oil by dividing the volume of the asphaltene non-solvent that causes the minimum optical density in the second crude oil by the initial volume of the second crude oil.

6. The method of claim 5, wherein a number i is added to the solubility ratio of the first crude oil and to the solubility ratio of the second crude oil, and wherein the number i is a number large enough to make the solubility ratio of the first crude oil and the solubility of the second crude oil both non-negative.

7. The method of claim 5, wherein the asphaltene non-solvent comprises a normal paraffin.

8. The method of claim 5, wherein the asphaltene non-solvent comprises n-pentane, n-hexane, n-heptane, n-octane, or any mixture thereof.

9. The method of claim 5, wherein the asphaltene non-solvent is n-pentane, and wherein the predetermined cutoff is 1.2.

10. The method of claim 5, wherein the minimum optical density is measured at a wavelength of about 1,400 nm to about 1,725 nm.

11. The method of claim 1, wherein the sum of the first value and the second value is less than the predetermined cutoff, and wherein modifying the composition of the proposed crude oil blend comprises:
replacing at least a portion of the second crude oil with a different crude oil;
adjusting a volume percent of the second crude oil relative to a volume percent of the first crude oil;
adding an additional crude oil that is different than the first crude oil and the second crude oil;
adding a compatibility additive to the crude oil blend; or
any combination thereof.

12. The method of claim 1, further comprising prescreening the second crude oil to determine whether or not to include the second crude oil, wherein the prescreening comprises:
combining the first crude oil and the second crude oil to produce the proposed crude oil blend;
measuring a precipitation propensity of the proposed crude oil blend;
comparing the measured precipitation propensity of the proposed crude oil blend to the sum of the first value and the second value; and
excluding the second crude oil if the measured precipitation propensity of the proposed crude oil blend differs from the sum of the first value and the second value by more than 20%.

13. The method of claim 1, wherein at least one of the first crude oil and the second crude oil comprises at least 0.17 wt % of asphaltenes.

14. A method for selecting crude oils to prepare a crude oil blend, comprising:
proposing an amount of a first crude oil and an amount of at least one second crude oil for combining with one another to produce a proposed crude oil blend, wherein at least one of the first crude oil and the second crude oil comprise asphaltenes;
measuring a precipitation propensity of the first crude oil and the second crude oil;
multiplying the precipitation propensity of the first crude oil by a volume percent of the first crude oil in the proposed crude oil blend to provide a first value;
multiplying the precipitation propensity of the second crude oil by a volume percent of the second crude oil in the proposed crude oil blend to provide a second value;
adding the first value and the second value to provide a sum for the proposed crude oil blend; and
combining the first crude oil and the second crude with one another to produce the proposed crude oil blend if the sum for the proposed crude oil blend is equal to or greater than a predetermined cutoff; or
modifying a composition of the proposed crude oil blend if the sum for the proposed crude oil blend is less than the predetermined cutoff to produce a modified proposed crude oil blend, wherein a sum for the modified proposed crude oil blend is equal to or greater than the predetermined cutoff.

15. The method of claim 14, wherein the precipitation propensity of the first crude oil and the precipitation propensity of the second crude oil are solubility ratios, wherein the solubility ratio for each of the first crude oil and the second crude oil is determined by:
adding an asphaltene non-solvent to a volume of the first crude oil and a volume of the second crude oil;
measuring a volume of the asphaltene non-solvent that causes a minimum optical density by near infrared spectroscopy for each of the volume of the first crude oil and the volume of the second crude oil;
dividing the volume of the asphaltene non-solvent that causes the minimum optical density in the first crude oil by the volume of the first crude oil to provide the solubility ratio of the first crude oil; and
dividing the volume of the asphaltene non-solvent that causes the minimum optical density in the second crude oil by the volume of the second crude oil to provide the solubility ratio of the second crude oil.

16. The method of claim 15, wherein:
a number i is added to the solubility ratio of the first crude oil and to the solubility ratio of the second crude oil,
the number i is a number large enough to make the solubility ratio of the first crude oil and the solubility of the second crude oil both non-negative,
the asphaltene non-solvent comprises n-pentane, and
the predetermined cutoff is 1.2.

17. The method of claim 15, wherein:
the first crude oil and the second crude oil are combined with one another to produce the proposed crude oil blend,
the second crude oil does not comprise asphaltenes, and
the precipitation propensity for the second crude oil, when blended with the first crude oil, is back calculated using a measured precipitation propensity of the proposed crude oil blend and the precipitation propensity of the first crude oil.

18. A method for selecting crude oils to prepare a crude oil blend, comprising:
proposing an amount of a first crude oil and an amount of at least one second crude oil for combining with one another to produce a proposed crude oil blend, wherein at least one of the first crude oil and the second crude oil comprise asphaltenes;
measuring a solubility ratio of the first crude oil and a solubility ratio of the second crude oil, wherein the solubility ratio for each of the first crude oil and the second crude oil is measured by:
adding to a volume of each of the first crude oil and the second crude oil a normal paraffin;
measuring a volume of the normal paraffin that causes a minimum optical density by near infrared spectroscopy for each of the first crude oil and the second crude oil;
dividing the volume of the normal paraffin that causes the minimum optical density in the first crude oil by the volume of the first crude oil to provide the solubility ratio of the first crude oil; and
dividing the volume of the normal paraffin that causes the minimum optical density in the second crude oil by the volume of the second crude oil to provide the solubility ratio of the second crude oil;
multiplying the solubility ratio of the first crude oil by a volume percent of the first crude oil in the proposed crude oil blend to provide a first value;
multiplying the solubility ratio of the second crude oil by a volume percent of the second crude oil in the proposed crude oil blend to provide a second value;
adding the first value and the second value to provide a sum for the proposed crude oil blend; and
combining the first crude oil and the second crude oil with one another to produce the proposed crude oil blend if the sum for the proposed crude oil blend is equal to or greater than a predetermined cutoff; or
modifying a composition of the proposed crude oil blend if the sum for the proposed crude oil blend is less than the predetermined cutoff to produce a modified proposed crude oil blend, wherein a sum for the modified proposed crude oil blend is equal to or greater than the predetermined cutoff.

19. The method of claim 18, wherein:
at least one of the first crude oil and the second crude oil comprises at least 0.17 wt % of asphaltenes,
the one normal paraffin comprises n-pentane, n-heptane, or a mixture thereof,
the minimum optical density is measured at a wavelength of about 1,550 nm to about 1,650 nm,
the composition of the proposed crude oil blend is modified to produce the modified proposed crude oil blend, and
modifying the composition of the crude oil blend comprises:
replacing at least a portion of the second crude oil with a different crude oil;
adjusting a volume percent of the second crude oil relative to a volume percent of the first crude oil;
adding an additional crude oil that is different than the first crude oil and the second crude oil;
adding a compatibility additive to the crude oil blend; or
any combination thereof.

20. The method of claim 18, wherein:
the first crude oil and the second crude oil are combined with one another to produce the proposed crude oil blend,
the second crude oil does not comprise asphaltenes, and
the precipitation propensity for the second crude oil, when blended with the first crude oil, is back calculated using a measured precipitation propensity of the proposed crude oil blend and the precipitation propensity of the first crude oil.
